(19) United States
(12) Patent Application Publication
(10) Pub. No.: US 2016/0242710 A1
(43) Pub. Date: Aug. 25, 2016

(54) PATIENT POSITION CONTROL FOR COMPUTED TOMOGRAPHY DURING MINIMALLY INVASIVE INTERVENTION

(71) Applicant: Siemens Aktiengesellschaft, Munich (DE)

(72) Inventors: Sasa Grbic, Princeton, NJ (US); Razvan Ionasec, Nuernberg (DE); Stefan Reichelt, Bamberg (DE)

(21) Appl. No.: 14/628,374

(22) Filed: Feb. 23, 2015

(51) Int. Cl. A61B 6/12; A61B 6/04; A61B 6/00; A61B 6/03

(52) U.S. Cl. CPC A61B 6/12; A61B 6/032; A61B 6/0407; A61B 6/461; A61B 6/485; A61B 6/5205; A61B 6/035

(57) ABSTRACT

Computed tomography is used during a minimally invasive intervention. A processor detects the inserted device (e.g., catheter) from a CT scan of the patient. The bed and/or gantry of the CT are moved based on the detected location of the inserted device. As the device is moved within the patient, the automatic detection is used to continue to adjust the CT field of view to scan the device. To further assist the intervention, combining scans with different fields of view relative to the patient may generate an extended field of view image.

### BACKGROUND

The present embodiments relate to computed tomography (CT). In particular, the present embodiments relate to use of computed tomography scanning in a minimally invasive intervention.

CT is a well-established modality in radiology for diagnosis and treatment planning. CT provides fast acquisition times for 3D volumes, has a high spatial resolution, and distinguishes bone and vascular structures with contrast agent. However, CT has a limited field of view-FOV, such as around 6 cm for real-time operation. This limited field of view is challenging for guidance of minimally invasive procedures. The interventional device is inserted at one location and navigated to the affected anatomy at another location. The device moves out of the CT field of view. This circumstance prevents the adoption of CT scanners in clinical practice as a common modality for interventional guidance.

### BRIEF SUMMARY

By way of introduction, the preferred embodiments described below include methods, systems, instructions, and computer readable media for use of computed tomography during minimally invasive intervention. A processor detects the inserted device (e.g., catheter) from a CT scan of the patient. The bed and/or gantry of the CT are moved based on the detected location of the inserted device. As the device is moved within the patient, the automatic detection is used to continue to adjust the CT field of view to scan the device. To further assist the intervention, combining scans with different fields of view relative to the patient may generate an extended field of view image.

In a first aspect, a method is provided for use of computed tomography during minimally invasive intervention. A first region of a patient is scanned with a computed tomography scanner while the patient is on a bed and while a minimally invasive object is within the patient. The minimally invasive object is detected in data resulting from the scanning of the first region. A position of the bed, of a gantry of the computed tomography scanner, or both the bed and the gantry is altered based on a location of the detected minimally invasive object. A second region of the patient different than the first region is scanned due to the altering.

In a second aspect, a computed tomography system is provided for use during a minimally invasive intervention. A gantry is operable to rotate an x-ray source and x-ray detector about a patient. A bed extends through the gantry. A processor is configured to detect a device inserted into the patient from information from the x-ray detector and to control movement of the bed, the gantry, or the bed and the gantry so that the device is positioned relative to the x-ray source and the x-ray detector.

In a third aspect, a method is provided for use of computed tomography during minimally invasive intervention. A computed tomography system detects an end of a device inserted into a patient. A patient bed or gantry is adjusted based on a position of the end. Data is acquired with the computed tomography system after the adjusting. An image of the patient and the end of the device is generated from the data.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for use of computed tomography during minimally invasive intervention;

FIG. 2 is a flow chart diagram of one embodiment of a method for use of computed tomography during minimally invasive intervention;

FIG. 3 illustrates a CT scanner and corresponding field of view, according to one embodiment;

FIGS. 4 and 5 show example images for detection of an interventional device;

FIG. 6 illustrates an example of bed movement to maintain an interventional device in a CT field of view;

FIG. 7 illustrates an example of gantry movement to maintain an interventional device in a CT field of view; and

FIG. 8 illustrates an example extended field of view for imaging.

### DETAILED DESCRIPTION OF THE DRAWINGS AND PRESENTLY PREFERRED EMBODIMENTS

Real-time guidance of a minimally invasive procedure is provided with a CT scanner. To continue to image the interventional device to assist in navigation, the table holding the patient or the gantry is automatically adjusted. Automatic adjustment of the patient table or scanner gantry based on detection of the interventional device may overcome the limited field of view CT. By detecting the device (e.g., catheter tip) in CT in real time and readjusting the patient table or gantry automatically based on the detection, the field of view is repositioned in order to guide the intervention over a longer distance. In addition, a compounded (e.g., larger field of view) image may be constructed and updated during the procedure.

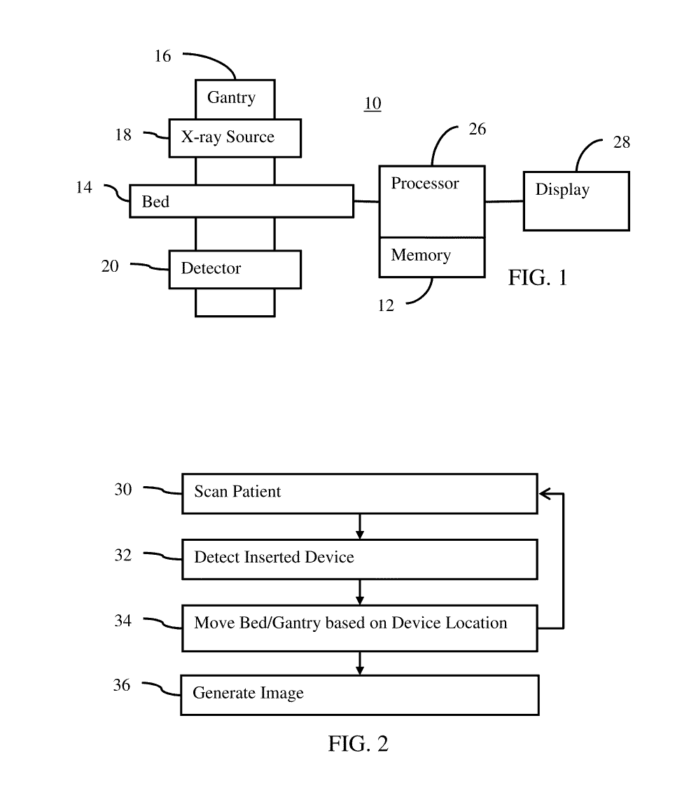

FIG. 1 shows a system 10 for use during a minimally invasive intervention. The system 10 includes a memory 12, a bed 14, a gantry 16 with an x-ray source 18 and detector 20, a processor 26, and a display 28. The system is a CT system or CT scanner. The processor 26, memory 12, and/or display 28 are part of a medical imaging CT system. Alternatively, the processor 26, memory 12, and/or display 28 are part of an archival and/or image processing system, such as associated with a medical records database workstation or server. In other embodiments, the processor 26, memory 12, and/or display 28 are a personal computer, such as desktop or laptop, a workstation, a server, a network, or combinations thereof.

Additional, different, or fewer components may be provided. For example, a network or network connection is provided, such as for networking with a medical imaging network or data archival system. As another example, a preoperative imaging system, such as a computed tomography or magnetic resonance imaging system, is provided. In another example, a user interface is provided.

The gantry 16, bed 14, x-ray source 18, and detector 20 provide a CT scanner. Any now known or later developed CT scanner may be used. The gantry 16 is a cylindrical structure with a motor, gears, and bearings or other structure for rotating the x-ray source 18 and detector 20 about the bed 14. The bed 14 supports the patient and extends through a hole formed by the gantry 16. The x-ray source 18 and detector 20 are separated by a region into which the patient is positioned.

The detector 20 detects X-rays from the source 18 passing through the patent. By transmitting X-rays through the patient to the detector 20, a projection image is provided. Any tissue, bone, catheter, other medical device, and/or any contrast agent along the path of travel of the X-ray beam interacting with the X-rays cause a detectable difference in intensity at the detector. Since each pixel or location of the detector represents an accumulation of responses along the path of travel, the fluoroscopy image is a projection image of the region. By scanning the patient from different angles due to the movement by the gantry 16 of the source 18 and detector 20, the x-ray attenuation or other patient characteristic at different locations distributed in three-dimensions may be reconstructed.

Due to the size of the gantry 16, x-ray source 18, and detector 20, only a portion of the patient is scanned at a given time. The patient may be scanned from different angles, but for the same region along the longitudinal axis of the patient and bed 14. The region is of any size, such as 10 cm or less, 6 cm, smaller, or larger along the longitudinal axis. Without moving the gantry 16, source 18 and detector 20, or bed 14, the region of the patient that may be scanned is limited. A larger region may be scanned by moving the patient relative to the x-ray source, but such scans take time that may not be available during an interventional procedure.

The reconstructed data from a scan of a given region includes response from the catheter or other inserted device, if inserted into the region of the patient being scanned. For example, during intervention, a catheter may be positioned within the patient. One or more fluoroscopy images are generated in real-time during the interventional procedure. The fluoroscopy image represents the vessel, catheter, other medical device, and/or other anatomy in the patient at the region.

The memory 12 is a graphics processing memory, a video random access memory, a random access memory, system memory, random access memory, cache memory, hard drive, optical media, magnetic media, flash drive, buffer, database, combinations thereof, or other now known or later developed memory device for storing data or video information. The memory 12 is part of an imaging system, part of a computer associated with the processor 26, part of a database, part of another system, a picture archival memory, or a standalone device.

The memory 12 stores data representing a region of a patient. The region is a three-dimensional region. The region is of any part of the patient, such as a region within the chest, abdomen, leg, head, arm, or combinations thereof.

A plurality of projections from the x-ray source 18 through the patient to the detector 20 from different angles relative to the patient is stored in the memory. Alternatively or additionally, a CT reconstruction of a volume representing the region is stored. The CT reconstruction determines voxel values (e.g., attenuation) distributed in three dimensions from the projections. The data is CT or x-ray data. The data may also include information representing an interventional device while in the region. The data is in any format, such as scalar intensities or attenuation values, image values, display or pixel mapped values, values on a regular three-dimensional grid (e.g., voxels), DICOM data, or other value format.

Frames of data representing different regions of the patient may be stored by the memory 12. As the relative position of the gantry and patient changes, different regions of the patient are scanned. The different regions may or may not overlap with other regions.

The memory 12 stores other information, such as a matrix or matrices for machine-learnt classifiers, calculated feature values, filtered or other image processed information, calibration values, and/or position information. Alternatively, scan data or other information is transferred to the processor 26 without storage in the memory 12 or with storage in another memory.

The memory 12 or other memory is alternatively or additionally a computer readable storage medium storing data representing instructions executable by the programmed processor 26 for use of a CT system in minimally invasive intervention. The instructions for implementing the processes, methods and/or techniques discussed herein are provided on non-transitory computer-readable storage media or memories, such as a cache, buffer, RAM, removable media, hard drive or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 26 is a general processor, central processing unit, control processor, controller, graphics processor, digital signal processor, three-dimensional rendering processor, image processor, application specific integrated circuit, field programmable gate array, digital circuit, analog circuit, combinations thereof, or other now known or later developed device for detecting an inserted device from CT scan data, moving the patient relative to the CT scanner, and controlling the scanning of the patient. The processor 26 is a single device or multiple devices operating in serial, parallel, or separately. The processor 26 may be a main processor of a computer, such as a laptop or desktop computer, or may be a processor for handling some tasks in a larger system, such as in an imaging system. In one embodiment, the processor 26 is a general processor for detecting the inserted device, and a controller for controlling a position of the bed 14 and/or gantry 16 and for controlling scanning.

The processor 26 is configured by instructions, design, hardware, firmware and/or software to be able to perform the acts discussed herein. FIG. 2 shows an example method implemented, at least in part, by the processor 26. The processor 26 causes scanning by the CT scanner in act 30, detects the inserted device in act 32, causes control of the bed and/or gantry position in act 34, and generates the image in act 36. The CT scanner performs acts 30 and 34. Different components may perform different ones of the acts. The processor 26 and/or CT scanner may perform different, additional, or fewer acts than shown in FIG. 2.

In one embodiment, the processor 26 is configured to detect a device inserted into the patient from information from the x-ray detector 20. From the detection, the processor 26 determines a position of the inserted device relative to the CT scanner. The movement of the bed, the gantry, or the bed and the gantry is controlled, such as by control signals from the processor 26 or by information provided by the processor 26 to a controller. The movement is performed so that the device is positioned relative to the x-ray source 18 and the x-ray detector 20, such as centering a part of the device in the scan region. The processor 26 causes scans of the patient with the device centered or positioned in the scan regions in an on-going basis. The detection and movement are repeated as more scans are performed, allowing the scanning to track or move with the inserted device. The movement is controlled to maintain the device in the scan region, such as centered in the scan region.

Since the inserted device is tracked, CT data (e.g., CT Fluoroscopy) is available for imaging the device in the patient. The processor 26 is configured to generate an image of the patient. The image includes response from the inserted device. Response from any injected contrast agent may also be included. The image represents the scan region, so includes response from tissue. Where scanning is repeated for different regions as the inserted device moves in the patient, the image may be replaced with an image representing the current region in an on-going manner.

In one embodiment, an extended field of view image is generated. The processor 26 combines the scan data, such as CT reconstructed data. The relative positions of the scan regions is indicated by the CT scanner and/or derived from data processing (e.g., finding a translation with a greatest similarity to define the overlap). The positions are used to combine the data from the different regions or regions corresponding to the x-ray detector 20 at different relative positions with the bed 14. The data is combined as projection images or combined as reconstructed voxel data. As the scan region changes, the image is updated to include the additional data received from the x-ray detector 20. The image shows the inserted device, including the path along which the device has traveled.

The display 28 is a monitor, LCD, projector, plasma display, CRT, printer, or other now known or later developed devise for outputting visual information. The display 28 receives images, graphics, or other information from the processor 26 or memory 12 and is configured to display the image, graphics, or other information.

FIG. 2 shows a method for use of computed tomography during minimally invasive intervention. The method is implemented by the system 10 of FIG. 1 or another system. The acts are performed in the order shown or other orders. Additional, different, or fewer acts may be provided. For example, the imaging of act 36 is not provided. As another example, acts for configuring the CT scanner, preparing the patient, and/or performing the intervention are provided.

The method is for positioning a CT scan region during a minimally invasive medical intervention. In such interventions, one or more devices are inserted into the patient. For example, a needle is inserted to inject a drug at a specific location or for biopsy. As another example, a catheter is inserted into a vein and pushed through the circulatory system to a location of interest for imaging, treatment (e.g., balloon expansion), drug injection, biopsy, or placement of an object (e.g., stenting). Any device used for intervention in cardiology, oncology, neurology, or other medical field is inserted into the patient. For example, any device currently guided with Fluoroscopic data using C-arm systems is inserted. Instead of using a C-arm system, the CT system may be used despite the limited field of view. Using the position control, needle, scanning provides catheter or other guidance for the medical professional so that the device (e.g., needle or catheter tip) is always in focus.

In act 30, the patient is scanned. The CT scanning is of any type. In one embodiment, a CT Fluoroscopic scan is performed. A CT scanner scans a region of the patient. With the bed 14 and gantry 16 in a given longitudinal location relative to the patient, one or more projections are obtained. An x-ray source and detector on the gantry may be rotated by the gantry about the patient. At different rotations or angles relative to the patient, a projection is obtained. In alternative embodiments, a projection is obtained from only one angle. In other embodiments, the bed and/or gantry continue to move while the region is scanned.

During the scan, the patient is on the bed. The minimally invasive object, such as the needle or catheter, is within the patient. For an initial scan, the operator may manually position the CT scanner to scan the patient at the location of insertion of the device. Alternatively, the CT scan region is positioned over a part of the body of interest where the device is believed to be located.

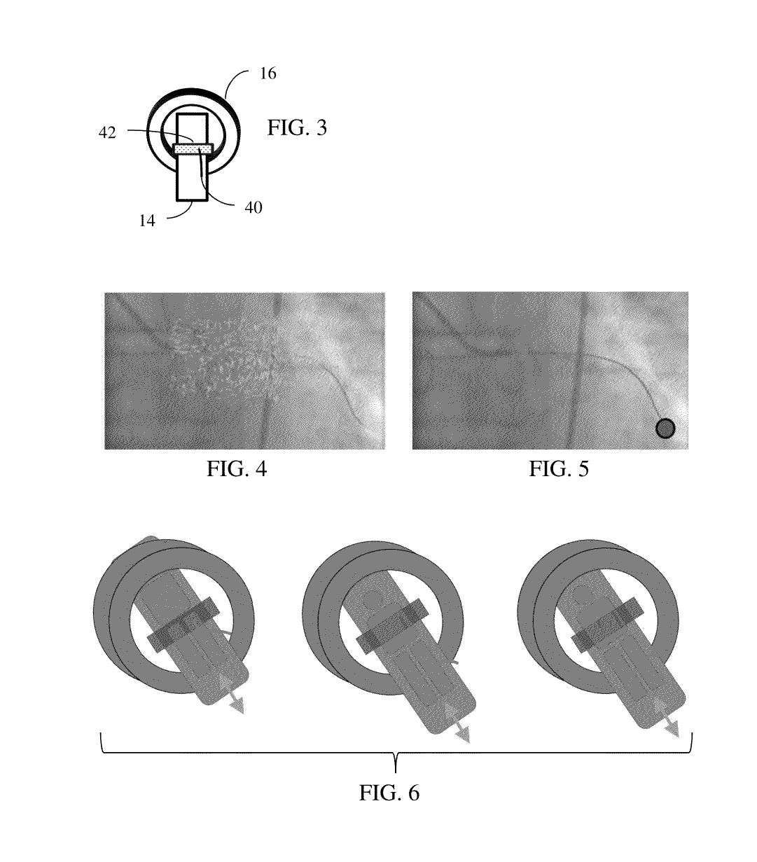

For the scan, projection data is acquired for a given cross-sectional region of the patient. The axial or longitudinal extent of the region is limited by the CT scanner, such as being less than 10 cm. For example, CT Fluoroscopy data containing the device to be monitored is acquired for the limited field of view of around 6 cm. FIG. 3 shows an example. The region 42 is positioned to include the tip of the catheter 40 while within the patient or as being inserted.

For a given scan, one or more projections are acquired. Each projection is a frame of data from the detector. Where projections for a given region are acquired from different angles, CT may be applied to reconstruct the volume of the region. Voxels in any grid format (e.g., Cartesian coordinate) are determined. A frame of data of the reconstructed information represents the volume. The frames of data representing anatomy of a patient with an inserted device are acquired by scanning. The anatomy, such as one or more vessels, may or may not include contrast agent. Since the inserted device is denser than tissue, such as due to including metal or hard plastic, the intensity for the denser medical instrument materials is higher than for surrounding soft tissue. The device may be located in the frame of data.

In act 32, a processor or CT scanner detects the minimally invasive object (i.e., device) as represented in the data resulting from the scanning of the region. A server or other remote processor in communication with the CT scanner may provide the detection.

The device is detected by processing the data resulting from the scan. One or more frames of data are processed to detect the device. The previously acquired data from the scan is used to detect. The detection is performed from projection data or from CT reconstructed data.

Any part of the device is detected. In one embodiment, the entire device in the region is detected. The tip or other part of the device may additionally be determined. The end of the device inserted into the patient is found. Alternatively, just the tip or other portion is detected without detecting the remaining part of the device in the region.

The device may include one or more fiducials. A metal or other marker more highly visible and/or with a specific pattern visible in x-ray imaging is provided. The detection uses pattern matching or other process to find the fiducial or fiducials. The parts of the device of interest have a known spatial relationship with the fiducial or fiducials.

Any other detection may alternatively or additionally be used. For example, filtering is applied to isolate information from the device. As another example, a threshold is applied. Edge detection or other segmentation may be used.

In one embodiment, a machine-learnt classifier detects the device. The processor extracts features from the data, such as steerable features or Haar wavelet features. The feature values are input to a matrix or matrices embodying the machine-learnt classifier so that the processor outputs an indication of whether the device is represented or at which locations the device is located.

Any machine learning may be used, such as a probabilistic boosting tree, Bayesian network, and/or neural network. A processor learns from training data to create the classifier. A training set of many example sets of data with known truth of the location of the device are used to derive the matrix mapping input features to an output classification. Machine-learnt classification may be robust to noise and handling objects with large variation. Since the training data also includes non-objects (e.g., tissue other than the device), the trained measurement models are robust to background noise.

In one embodiment, a geometric model of the catheter is extracted from fluoroscopy or other CT data using a machine-learnt classifier. For example, a likelihood of a voxel being part of the catheter is calculated using a machine-learned classifier. FIG. 4 shows a fluoroscopy projection image from a CT scan. The locations with higher probabilities of being part of the catheter are highlighted. Based on the likelihood data output by the first classifier and the original image or CT data, another classifier locates the catheter tip. For example, the tip is detected using a probabilistic boosting tree created classifier and Haar features computed both on the original image and the derived likelihood “probability” image from the first classifier. In alternative embodiments, the tip classifier does not use the probability information as an input. Instead, the output of the tip classifier is probabilistically combined with the likelihood output of the first classifier. Filtering, connected component, and/or edge detection may be used to refine the detection results. FIG. 5 shows a detected tip of a catheter and detected catheter in an image. Other machine learning approaches may be used, including using just one classifier.

In act 34, a position of the bed, of a gantry, or both the bed and the gantry of the computed tomography scanner are altered. The bed includes a motor, drive and rail, chain and pulley, robotic arm, and/or other device for moving the patient along the longitudinal axis through the gantry. Alternatively or additionally, the gantry includes a motor and other devices (e.g., drive and rail) for moving along the longitudinal axis relative to the bed. A controller sends signals to control the position or movement of the bed and/or gantry.

The movement positions the x-ray source and detector or scanning part of the CT scanner to scan a different region of the patient. The different region to be scanned due to the movement may overlap with one or more previous scan regions. For example, a 6 cm longitudinal field of view is provided. By moving the bed 2 cm, a new region is to be scanned that overlaps the previous region by 4 cm, but includes 2 cm of the patient not scanned in the immediately previous scan.

Either the patient table or the gantry moves to a different position. For example, the bed moves relative to the gantry to position a different region of the patient for scanning. The bed moves the patient. FIG. 6 shows an example. In a first position of the bed, the upper thighs of the patient are in the scan region. In another position, the middle of the torso is in the scan position. In yet another position, a lower portion of the torso is in the scan position. The three examples of FIG. 6 show a sequence with or without intervening regions being scanned. The patient table automatically adjusts during the sequence.

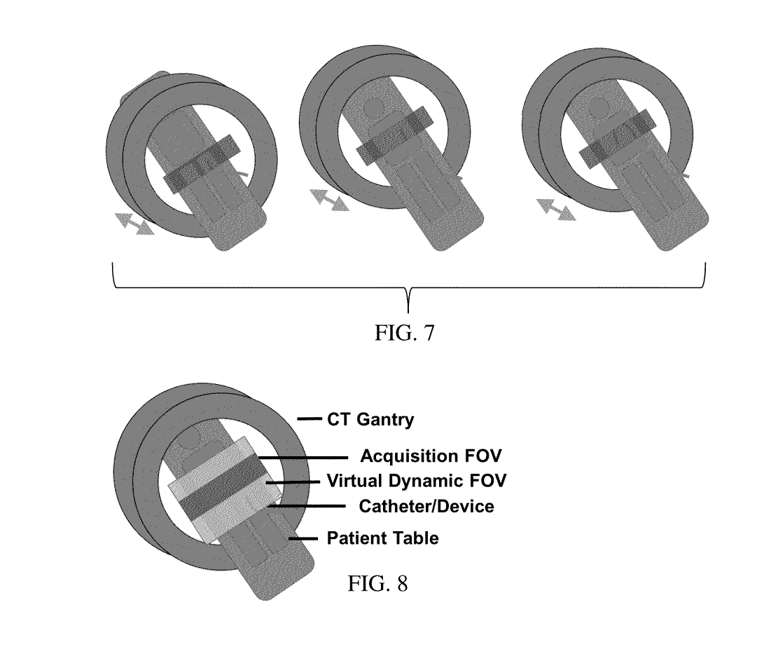

Alternatively or additionally, the gantry moves relative to the patient. For example, the gantry moves along the longitudinal axis to position the x-ray source and detector around a different part of the bed and patient. The patient remains stationary, relative to the room, while the gantry moves. FIG. 7 shows an example. A sequence of different gantry positions and resulting scan regions relative to a patient are shown. This is the same region sequence shown in FIG. 6, but is achieved by gantry movement rather than bed movement.

The movement is based on a location of the detected device. The detection indicates a location in the region represented by the data. Since the CT scanner is positioned relative to that region, detection of the device indicates a location of the device relative to the CT scanner. The location is used to alter the region.

Any criterion may be used for altering the region to be scanned. The region may be positioned to include more of the device, such as placing a tip at an edge of the region with or without margin. The tip may be positioned out of the region to position another part of the device in the region. Another part of the device may be centered in the region or positioned at the edge. Any part or section of the device may be placed in any location relative to the region.

In one embodiment, the bed and/or gantry are moved to place the tip of the device, such as a tip of a catheter, in a center of the scan region of the computed tomography scanner. To subsequently scan the patient, the bed or gantry is moved so that the tip as detected from the last or temporally most immediate scan is centered. Where detection and movement happens sufficiently quickly (e.g., within seconds) relative to the pace of movement of the device in the patient, the tip should still be within the scan region when the subsequent scan with the new region position is performed. In other embodiments, the scan region is placed so that the tip will likely be at the center when the scan occurs. A rate of previous or expected movement is used to predict the location of the tip at a time of a future scan based on a previous location of the tip. The region is positioned based on the prediction.

This alteration of the position of the region relative to the patient compensates for the small field of view of the CT Fluoroscopy acquisition by continuously adjusting the field of view so that the catheter tip is located at the center of volume. Based on the catheter detection, either the patient table or the gantry is realigned so that the updated image is centered on the catheter. FIGS. 6 and 7 show an end of the catheter being in each region. The bed and/or gantry move in either direction relative to the patient to maintain the catheter tip within the scan region over time.

Referring again to FIG. 2, a feedback from act 34 to act 30 is shown. After moving the bed or gantry in act 34, the scan of act 30 is repeated. The detection of act 32 is repeated and the bed or gantry is moved again based on the device location. In one or more repetitions, the region may stay at the same location. By repeating the detection, adjustment, and acquisition of scan data as the device moves through the patient during the intervention, the device continues to be automatically imaged or scanned without the physician having to manually adjust the position of the patient relative to the scanner. The process continues until or after the device reaches the region of the affected anatomy. The device is tracked until and/or through treatment or use of the device at the desired location in the patient.

In act 36, an image is generated. The CT scanner or processor generates an image from the scan data. The image is of the patient with or without the device. For example, the image is of the patient and includes the end of the device.

The image is generated from projection data or reconstructed data. The image represents the region of the patient from a given scan (e.g., one or more x-ray generation events and detections with the bed and gantry in a given relationship or position). For reconstruction data, any three-dimensional rendering may be used, such as surface or projection rendering. Planar reconstruction from the volume representing data may be used, such as using data along a plane to represent that plane of the patient in the image.

In one embodiment, an extended field of view is generated. As the device moves in the patient and the CT scanner acquires data from different regions of the patient, data representing an overall region extending beyond any given single region of the CT scanner is provided. The data is combined to form an extended field of view. The data resulting from repetition of the scanning while tracking the device is combined. Data from different relative positions of the x-ray source and detector with the bed is combined.

The positions of the regions are known from the CT scanner. Using the position sensors of the bed and/or gantry with or without calibration adjustments, the relative longitudinal locations represented by the different frames of data is known. The combination of data from the frames is spatially aligned using the relative position information.

Alternatively or additionally, the frames of data are translated in increments and a similarity between frames is calculated at each increment. The translation associated with the greatest similarity indicates the relative offset or position. In another approach, the frames of data from different scans are registered with a pre-operative scan of a larger region or entire patient. The registration indicates the relative position in the pre-operative data. The relative positions from different CT scans during device navigation are used to combine.

Any combination may be used to stitch the frames or images together. For example, data from different frames representing a same location are averaged. Minimum or maximum selection may instead be used. As another example, the most recently acquired data is used for any given location. A newly acquired frame of data replaces any data in the combination representing the same locations.

After combination, a frame of projection or volume (reconstructed) data is provided for the extended field of view. The locations represented in the frame are for more than the field of view possible for the CT scanner with the bed and gantry in one relative position. A larger region than a single scan region is represented.

In the frame of data for the extended field of view, the device is represented at different times. Since the path through which the device travels is the same, the temporal aspect blend together, representing the device in the extended field as along the entire path. Filtering or other process may be used to enhance and/or de-emphasize parts of the device relative to tissue or other anatomy represented in the frame of data.

FIG. 8 shows an example of the extended field of view. In the example of FIG. 8, three adjacent or connected regions with or without any overlap are shown together. Scan data from the three different regions is combined, providing an extended field of view. Since the extended field of view is built-up or repetitively imaged during the intervention, the field of view is dynamic. During the procedure, a virtual (i.e., extended) dynamic field of view is generated. A sequence of images representing the extended field of view over time is generated. As the procedure continues, the virtual field of view may grow larger. The image information or data prior to imaging is compounded to generate a large field of view image. If the same area is acquired a second time, this part of the compounded image may be automatically updated or replaced with the more recently acquired data.

The resulting image may assist with navigation of the inserted device. The extended field of view image shows the anatomy for the past and current path of the device. This information is used to continue to guide the device to the treatment, biopsy, or diagnosis location. CT scanning is used for the interventional procedure.

Data prior to image generation is compounded for the extended field of view. Alternatively, images are generated for each region, and the images are compounded.

Any process may be used to generate the extended field of view image. Where the frame of data is a combination of projections from a same angle relative to the patient, a two-dimensional projection image is generated. Where the frame of data for the extended field of view is a reconstruction of an extended volume, a projection from a given angle may be simulated. The voxels for the CT volume represent attenuation. By summing along virtual ray lines from a virtual x-ray source, the resulting attenuation for each pixel is created. The virtual source is at any desired position. The integrated data is mapped to display values and imaged as a projection image.

In other embodiments, three-dimensional rendering of the extended field of view is used. Projection rendering (e.g., minimum or maximum selection, alpha blending, or averaging) or surface rendering is performed. Maximum intensity projection is used in one embodiment. Multi-planar reconstruction may be used. One or more planes are defined in the volume. The voxels on or near the plane are used to create a two-dimensional image.

The frame of data for the extended field of view and/or the generated images may be further processed. For example, a simulated projection or fluoroscopic image may be processed to remove bone, cement material, or other more highly attenuating material. By removing the more highly attenuating material prior to generating the image, the resulting image may be more useful for guiding navigation.

The patient holds still and may or may not hold their breath during scanning. Alternatively, different scans are acquired with intervening patient motion from breathing. Heart motion may also occur. ECG, breathing monitor, or other motion detection may be used. The scans are triggered to occur at particular phases of the cyclical motion. For voluntary motion, motion correction may be applied. A transform indicating the measured motion may be applied to the data and/or to the position information for the region to counteract the motion. In alternative embodiments, images are generated without motion correction and/or without triggering.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for use of computed tomography during minimally invasive intervention, the method comprising:
scanning a first region of a patient with a computed tomography scanner while the patient is on a bed and while a minimally invasive object is within the patient;
detecting the minimally invasive object in data resulting from the scanning of the first region;
altering a position of the bed, of a gantry of the computed tomography scanner, or both the bed and the gantry based on a location of the detected minimally invasive object; and
scanning a second region of the patient different than the first region due to the altering.

2. The method of claim 1 wherein scanning the first region comprises scanning a cross-sectional region having an axial extent along the patient of less than 10 cm.

3. The method of claim 1 wherein scanning the first and second regions comprises performing computed tomography fluoroscopic imaging.

4. The method of claim 1 wherein scanning the first and second regions comprises rotating an x-ray source and detector on the gantry about the patient with the bed and gantry in first and second relative positions, respectively.

5. The method of claim 1 wherein the minimally invasive object comprises a needle or a catheter, and wherein detecting comprises detecting a tip of the needle or a tip of the catheter.

6. The method of claim 1 wherein detecting comprises detecting with a machine-learnt classifier.

7. The method of claim 1 wherein altering comprises moving the bed relative to the gantry.

8. The method of claim 1 wherein altering comprises moving the patient by moving the bed.

9. The method of claim 1 wherein altering comprises moving the gantry relative to the patient.

10. The method of claim 1 wherein altering comprises altering to place a part of the minimally invasive object at a center of a scan region of the computed tomography scanner for the scanning of the second region.

11. The method of claim 1 further comprising:
generating an image of the patient from both the data resulting from the scanning of the first region and data resulting from the scanning of the second region.

12. The method of claim 11 wherein generating the image comprises simulating an x-ray projection using a data set stitched together from the data resulting from the scanning of the first region with the data resulting from the scanning of the second region.

13. The method of claim 1 further comprising repeating the detecting, the altering, and scanning of additional regions as the minimally invasive object moves relative to the patient.

14. A computed tomography system for use during a minimally invasive intervention, the system comprising:
a gantry operable to rotate an x-ray source and x-ray detector about a patient;
a bed extending through the gantry; and
a processor configured to detect a device inserted into the patient from information from the x-ray detector and to control movement of the bed, the gantry, or the bed and the gantry so that the device is positioned relative to the x-ray source and the x-ray detector.

15. The computed tomography system of claim 14 wherein the processor is configured to cause scans of the patient with the device centered in scan regions for the scans, the movement controlled to maintain the device being centered in the scan region.

16. The computed tomography system of claim 14 wherein the processor is configured to generate an image of the patient from a combination of data from the x-ray detector with the bed and gantry at different relative positions, the combination of the data reconstructed by computed tomography; and
further comprising a display configured to display the image.

17. The computed tomography system of claim 16 wherein the processor is configured to update the image as additional data is received from the x-ray detector with the bed and gantry at different relative positions and the device at a different location in the patient.

18. A method for use of computed tomography during minimally invasive intervention, the method comprising:
detecting, with a computed tomography system, an end of a device inserted into a patient;
adjusting a patient bed or gantry based on a position of the end; and
acquiring data with the computed tomography system after the adjusting; and
generating an image of the patient and the end of the device from the data.

19. The method of claim 18 wherein the detecting is from prior data acquired with the computed tomography system.

20. The method of claim 18 wherein detecting, adjusting, and acquiring are repeated as the end of the device moves within the patient, and wherein generating the image comprises generating an extended field of view from the data acquired with the computed tomography system from a plurality of the repetitions.
